(19) United States
(12) Patent Application Publication
(10) Pub. No.: US 2024/0268663 A1
(43) Pub. Date: Aug. 15, 2024

(54) DEVICE AND METHOD FOR DIAGNOSIS, AND FOR PLANNING AND/OR MONITORING AN OPERATION ON THE EYE

(71) Applicant: Carl Zeiss Meditec AG, Jena (DE)

(72) Inventors: Wei-Jun CHEN, Jena (DE); Ralf EBERSBACH, Schmölln (DE); Roland BERGNER, Jena (DE); Marko SCHAAF, Jena (DE); Martin VOLKWARDT, Stadtroda (DE)

(21) Appl. No.: 18/568,151

(22) PCT Filed: May 20, 2022
(86) PCT No.: PCT/EP2022/063724

§ 371 Date: Dec. 7, 2023

(30) Foreign Application Priority Data
Jun. 8, 2021 (DE) 10 2021 205 738.1

(51) Int. Cl. A61B 3/117; A61B 3/00; A61B 3/10; A61B 3/15; G06T 5/70; G06T 7/00; G06T 7/13; G06T 7/62; G06T 7/73

(52) U.S. Cl. CPC A61B 3/1176; A61B 3/0041; A61B 3/0091; A61B 3/102; A61B 3/156; G06T 5/70; G06T 7/0012; G06T 7/13; G06T 7/62; G06T 7/73; G06T 2207/10024; G06T 2207/10101; G06T 2207/20208; G06T 2207/30041; G06T 2207/30204

(57) ABSTRACT

A light source that projects a fixation mark into the eve and an image sensor that receives light that has been reflected, diffracted or scattered from the cornea, lens and retina of the eve. A digital camera that captures HDR images of the eve predominantly only with activated fixing illumination. A control unit is designed to detect reflections, as well as diffracted and scattered light of the fixing mark of eve structures in the images transferred from the image sensor and to optimize, evaluate and display the captured HDR images of the eye on a display unit. The invention is provided for different ophthalmological devices, but, in principle, it can be applied to other technical fields in which additional information of the object to be imaged is generated and correspondingly evaluated via the capturing of HDR images.

### CLAIM TO PRIORITY

This application claims priority from Application PCT/EP2022/063724, filed May 20, 2022, and claims priority from DE Patent Application No. 10 2021 205 738.1, filed Jun. 8, 2021, each of which are incorporated by reference in their entireties in this application.

### TECHNICAL FIELD

Embodiments of the present invention relate to a device and a method for diagnosis, and for planning and/or monitoring the course of an operation on the eye, wherein the device includes an optical system for aligning and fixating the eye with respect to the optical axis of the device.

### BACKGROUND

In ophthalmology, observing and documenting the status of the eye probably represent the most frequently required step for diagnosing ailments, and for planning, performing and assessing operations on the human eye.

The known prior art has disclosed a multiplicity of examination and treatment apparatuses which are based on different principles. However, what applies to all types of such apparatuses is that, where possible, the eye is always aligned in the same way and stably in relation to the respective apparatus. In general, this requires a separate system for aligning and fixating the patient's eye.

In such an eye fixation system, a light source is used to project a fixation mark along the optical axis of the respective apparatus and into the human eye. The patient is asked to gaze at this fixation mark, whereby their eye is aligned with the optical axis of the apparatus. In a second step there is an assessment as to how stable the alignment of the patient's eye is.

In the simplest case, the reflection of the fixation mark on the cornea of the eye to be examined is used to this end. In this context, this so-called “first Purkinje reflex” is recorded and analyzed in respect of its alignment and/or possible movements in relation to the optical axis of the apparatus, and for further interaction between apparatus and patient.

Even though the assessment of the alignment of the examined eye with respect to the apparatus is the main object of the eye fixation system, further information regarding peculiarities of parts of the eye to be examined can be generated, by way of reflection, diffraction, and refraction of the fixation light, by the light source that generates the fixation mark.

In the apparatuses known from the prior art, only the evaluation of the first Purkinje reflex is envisaged for the fixation light, to thereby generate information with regard to the alignment or fixation of the eye to be examined.

### SUMMARY OF THE INVENTION

In this respect, FIG. 1 shows the image of an eye illuminated only by fixation light that is recorded with an intensity depth/color depth of 8 bits, with the bright spot representing the first Purkinje reflex from the cornea of the eye.

A disadvantage of these known apparatuses is that collection of further information generated by the fixation light by way of reflection, diffraction, and refraction is not envisaged.

Moreover, this information is suppressed by other illumination, for instance for a slit, a Placido ring system, a keratometric point pattern or else the ambient conditions, and is consequently generally dispensed with for the ophthalmological diagnosis.

Example embodiments of the present invention include the collection of additional information in respect of the eye to be examined and enable an improved diagnosis, and make possible for planning and/or monitoring the course of an operation on the eye.

An example embodiment of the invention includes a device for diagnosis, and for planning and/or monitoring the course of an operation on an eye, the device inter alia comprising an optical system for fixating the eye with respect to the optical axis of the device, consisting of a light source for projecting a fixation mark into the eye, an image sensor for receiving light reflected, diffracted or scattered by the cornea, lens, and retina of the eye, and a control unit designed to detect reflections, and light of the fixation mark diffracted and scattered by eye structures in the images transmitted by the image sensor, by virtue of the fact that a digital camera is present for recording HDR images of the eye, that the digital camera is designed to record HDR images of the eye predominantly only in the case of an activated fixation illumination, and that the control unit is further designed to optimize and evaluate the recorded HDR images of the eye and display these HDR images on the display unit.

“HDR images” (high dynamic range—HDR), “images with a high dynamic range”, or else “high-contrast images” should be understood to mean various techniques for recording and reproducing images with significant brightness differences above approximately 1:1000.

HDR images can be recorded directly by many cameras, can be generated from exposure series of photos with a normal dynamic range (low dynamic range—LDR), or can be calculated directly as 3-D computer graphics.

Example developments and configurations according to the invention relate to the digital camera, which has an intensity depth/color depth of more than 8 bits and which is designed as a telecentric imaging system.

In accordance with a further configuration, the device according to the invention may be designed as a retrofit unit for ophthalmological apparatuses.

However, it is also possible to configure the device according to the invention as an independent one-function apparatus for retro-illumination analysis for the eye of the patient.

An example embodiment of the invention includes a method according to the invention by virtue of the fact that the HDR images of the eye recorded by the digital camera predominantly only in the case of an activated fixation illumination are optimized, evaluated and displayed on the display unit by operation of the control unit, with known image processing and image optimization methods being used for the optimization and evaluation of the HDR images of the eye.

According to an example embodiment of the invention, the HDR images of the eye are optimized and evaluated on the basis of the following steps:
a) removing the image noise,
b) optimizing the intensity image representation in order to obtain a “moon-like” pupil image of the eye,
c) detecting the pupil edge and determining the diameter,
d) comparing the diameter with an expected value,
e) should the diameter be too small there is a dilation and a renewed performance of steps a) to d),
f) should the diameter at least correspond to the expected value there is a detection of the image content in respect of abnormalities.

Example embodiments of the invention can be used for different ophthalmological apparatuses for diagnosis, and for planning and/or monitoring the course of operations on the eye, which ophthalmological apparatuses inter alia comprise an optical system for fixating the eye with respect to the optical axis of the device.

In principle, however, the solution can also be used in other technical fields, in which additional information regarding the object to be imaged is generated and evaluated accordingly as a result of the use of a digital camera suitable for recording HDR images.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

The invention is described in more detail below on the basis of example embodiments. In this respect, some of the drawings show images of eyes illuminated only by fixation light, with the ring in the interior in each case specifying the lateral position of the first Purkinje reflex:

FIG. 1: depicts an image recorded with an intensity depth/color depth of 8 bits,

FIG. 2: depicts HDR images recorded with an intensity depth/color depth of 12 bits, with linear or optimized intensity imaging for 8-bit visualization,

FIG. 3: depicts HDR images of eyes with a small pupil,

FIG. 4: depicts HDR images of eyes with a cataract,

FIG. 5: depicts an HDR image of an eye with a cataract, for defining the direction of an OCT scan,

FIG. 6: depicts HDR images of eyes with a spherical IOL,

FIG. 7: depicts HDR images of eyes with a toric IOL,

FIG. 8: depicts HDR images of an eye with a particle moving in the tear film,

FIG. 9: is a box diagram according to an example embodiment of the invention, and

FIG. 10: is a flowchart of a method according to an example embodiment of the invention.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION

The proposed An example device for diagnosis, and for planning and/or monitoring the course of an operation on an eye, which inter alia comprises an optical system for fixating the eye with respect to the optical axis of the device, consists of includes a light source for projecting a fixation mark into the eye, an image sensor for receiving light reflected, diffracted or scattered by the cornea, lens, and retina of the eye, and a control unit designed to detect reflections, and light of the fixation mark diffracted and scattered by eye structures in the images transmitted by the image sensor.

According to example embodiments of the invention, a digital camera is present for recording HDR images of the eye and is designed to record HDR images of the eye predominantly only in the case of an activated fixation illumination. The control unit is further designed to optimize and evaluate the recorded HDR images of the eye and display these HDR images on a display unit.

Furthermore, the control unit is designed to monitor the alignment of the eye in relation to the optical axis of the device by detecting the fixation mark in the images transmitted by the image sensor, and to display said alignment on the display unit.

In this case, the digital camera has an intensity depth/color depth of more than 8 bits, for example 10 to 14 bits and in another example 16 bits or more.

Bits per pixel (bpp) are the number of bits with which a pixel is displayed. The number is dependent on the resolution and is a measure of the intensity depth/color depth.

Thus, an intensity depth with 256 intensities can be displayed with 8 bits. By comparison, an intensity depth in the high-intensity range with 65 536 intensities can already be recorded in the case of a resolution of 16 bits and an intensity with 16.7 million intensities can be recorded in the case of a 24-bit resolution. In what is known as a “true color” representation with 24 bpp, 8 bits are available for each RGB channel.

However, there are also a few representation techniques that operate at higher color resolutions. In these so-called “deep color” representations with 30, 36 or 48 bpp, 10, 12 or 16 bits are available for each RGB channel.

By way of example, a color resolution of 36 bpp is an extension of 24 bpp, in which each RGB channel has 8 bits available and there additionally are 8 bits available for an alpha channel for blending the display.

These representation techniques include a variant of HD representation with 1080 lines and a 4K standard of D-cinema. A further increase in the bits per pixel is possible with alpha blending. Novel technologies offer more options for displaying HDR images by way of suitable intensity imaging methods.

In accordance with a first example configuration of the device, the HDR digital camera is a constituent part of a telecentric imaging system.

A telecentric imaging system allows collection of the limited light in the vicinity of the image axis and provision of a distance-independent measurement for the required eye information, for example the pupil size.

In accordance with a second example configuration of the device, devices for protection against ambient light are present.

In accordance with a third, example configuration of the device, the device can be in the form of a retrofit unit for ophthalmological apparatuses or formed as an independent one-function apparatus for retro-illumination analysis for the eye of the patient.

In the example method for diagnosis, and for planning and/or monitoring the course of an operation on an eye, a fixation mark is projected into the eye by a light source, the light of the fixation mark reflected, diffracted, or scattered by the cornea, lens, and retina of the eye is imaged on an image sensor, and reflections, and light of the fixation mark diffracted and scattered by eye structures are detected by a control unit in the images transmitted by the image sensor.

According to the invention, HDR images of the eye are recorded by a digital camera predominantly only in the case of an activated fixation illumination and said HDR images are optimized, evaluated and displayed on the display unit by operation of the control unit.

Further the fixation mark in the images transmitted by the image sensor is detected by the control unit, and hence the alignment of the eye in relation to the optical axis of the device is monitored and displayed on a display unit.

In this case, the digital camera records HDR images of the eye with an intensity depth/color depth of more than 8 bits, for example 10 to 14 bits and in another example 16 bits or more.

In accordance with a first example configuration of the method, the HDR images of the eye are recorded telecentrically in order to collect the limited light in the vicinity of the image axis.

In accordance with a second example configuration of the method, the ambient light is suppressed for the purpose of recording the eye images.

The HDR images of the eye recorded by a digital camera are optimized, evaluated, and displayed by the control unit by application of known image processing and image optimization methods.

Since HDR images can be displayed directly on conventional monitors and media and/or only in restricted fashion, the brightness contrast must be reduced for display purposes. This procedure is referred to as tone mapping. Irrespective of this restriction, it is possible to avoid overexposures and underexposures, obtain better image details and carry out more far-reaching image processing when proceeding from HDR images. Such applications are advantageous, especially also in medicine.

Newer versions of some image processing programs can process HDR images directly. This makes it possible to carry out changes in brightness, contrast, and color, without this leading to losses in the form of saturated pixel values.

HDR images with a greater dynamic range of the pixel intensity offer the option of adaptively imaging pixel intensities on 8-bit monitor apparatuses, also by way of other methods than only linear imaging.

According to the invention, the HDR images of the eye are optimized and evaluated on the basis of the following steps:
a) removing the image noise,
b) optimizing the intensity image representation in order to obtain a “moon-like” pupil image of the eye,
c) detecting the pupil edge and determining the diameter,
d) comparing the diameter with an expected value,
e) should the diameter be too small there is a dilation and a renewed performance of steps a) to d),
f) should the diameter at least correspond to the expected value there is a detection of the image content in respect of abnormalities.

According to method steps a) and b), the recorded HDR images are improved by virtue of the image noise being removed and the intensity image representation being optimized to obtain a “moon-like” pupil image of the eye.

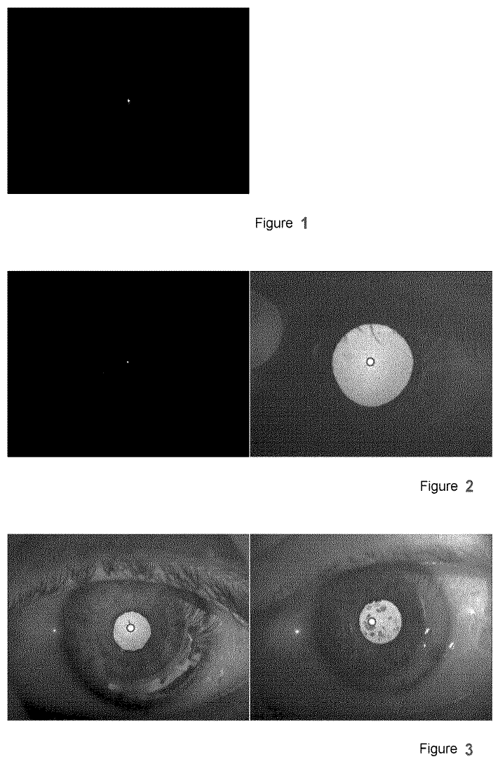

In this respect, FIG. 2 shows the HDR images recorded with an intensity depth/color depth of 12 bits, with linear or optimized intensity imaging for 8-bit visualization.

The image representations show HDR images of eyes which are illuminated only by fixation light. In this case, the left HDR image was visualized with linear intensity imaging and the right HDR image was visualized with optimized intensity imaging.

According to method steps c) and d) the recorded HDR images are processed and evaluated by virtue of the pupil edge being detected, the diameter being determined, and said diameter being compared with an expected value of the diameter.

In the next method step e) there is a dilation and a renewed performance of steps a) to d) should the diameter be too small.

Dilation (image processing) should be understood to mean a basic morphological operation in digital image processing, in which the original image is generally “expanded” or “extended” by use of a structuring element.

In this respect, FIG. 3 shows an HDR image of an eye with a small pupil.

According to the last method step f), the image content in the form of the intensity distributions of the recorded HDR images is detected in respect of abnormalities should a diameter of the pupil at least corresponding to the expected value be present.

In accordance with a further configuration of the method, the HDR images are detected in respect of dark or shadow regions present, which, in the region of the crystalline lens, indicate a possible cataract disease or posterior capsular opacification.

By way of appropriate analysis, classifying the stage of the cataract of the crystalline lens is optionally possible.

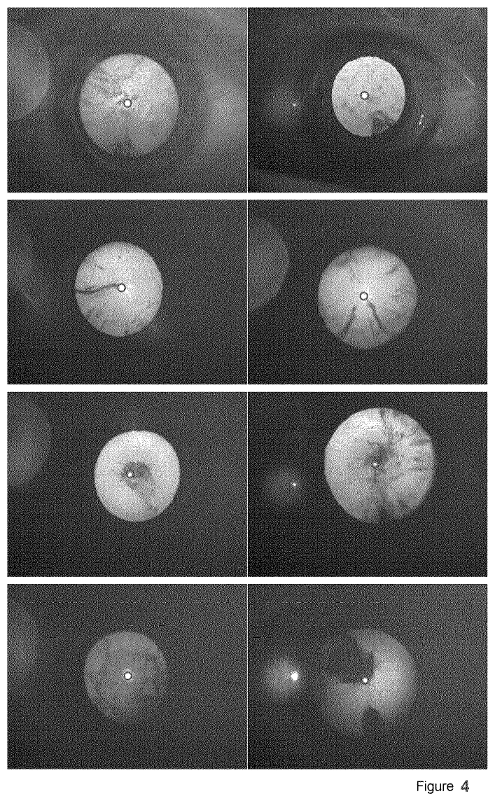

In this respect, FIG. 4 shows a number of HDR images of eyes with a cataract. In this respect, the visible dark or shadow regions indicate different stages of the cataract disease.

In this context, it is advantageous, for example, if the detected abnormalities may also lead to further clinical examinations of the eye, especially if a cataract disease is detected.

In particular, detected abnormalities under step f) may also lead to an OCT-B-scan being performed in accordance with the observed eye information, in order to determine the area/stage of the cataract and in order to obtain further clinically meaningful OCT depth information for the examined eye.

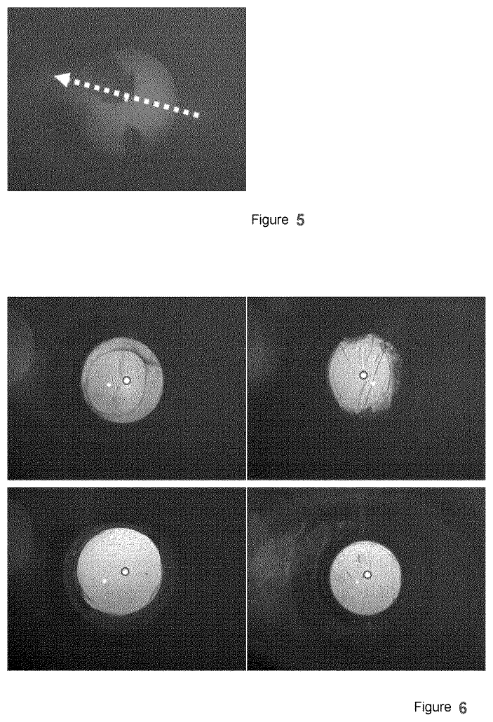

In this respect, FIG. 5 shows an HDR image of an eye with a cataract. The preferred direction for an additional OCT scan can easily be defined on the basis of the visible dark or shadow regions.

As a result, it is for example made possible to determine the region and/or stage of the cataract more precisely, and to obtain further clinically meaningful OCT depth information for the examined eye.

In accordance with a further configuration of the method, the edge of an IOL and/or the markers thereof present can also be determined when detecting abnormalities in the HDR images.

Optionally, this renders possible an analysis of the IOL status post-surgery on the basis of the observed IOL boundary in the HDR image.

Detected abnormalities under step f) may for example also be the 3rd and 4th Purkinje reflex, whereby it is possible to determine the tilt and centration of the IOL in the eye.

Furthermore, detected abnormalities under step f) for example also allow the size and quality of the capsulorhexis to be assessed.

In this respect, FIG. 6 shows HDR images of eyes with a spherical IOL.

Should the implanted IOL be a toric IOL, the detected markers can be used not only to determine the position thereof (decentration, axis alignment, etc.), but also the orientation thereof.

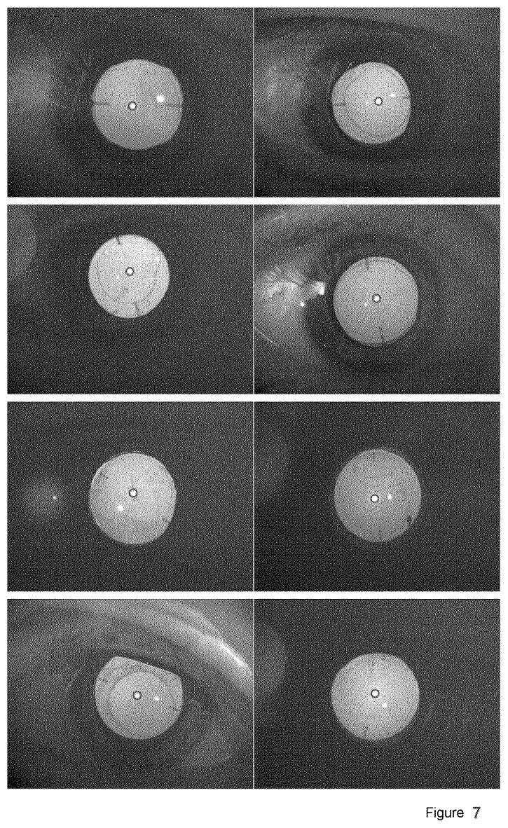

In this respect, FIG. 7 shows HDR images of eyes with a toric IOL.

In accordance with a further configuration of the method, a series of HDR images of the eye can be recorded, for example in order to be able to monitor the function of the tear film.

In this case, the recorded HDR images are analyzed in time-dependent fashion, with the movement of particles in the tear film in particular being detected.

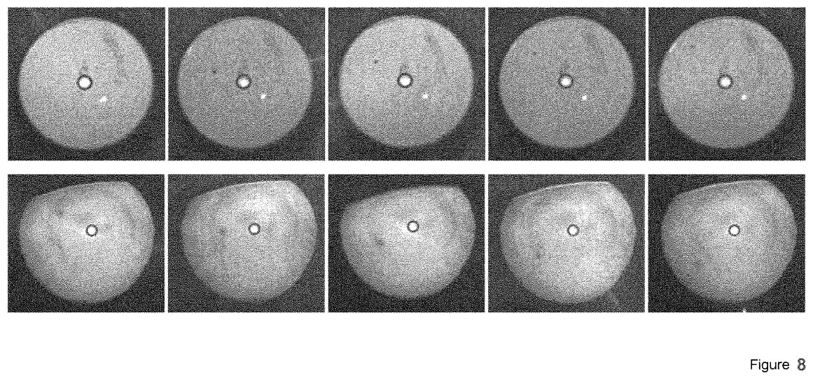

In this respect, FIG. 8 shows HDR images of an eye with a particle moving in the tear film.

In the HDR images, which were recorded at a frequency of 1 Hz, an upwardly moving dark point can be identified in the upper row and a downwardly moving dark point can be identified in the lower row.

The arrangement according to the invention provides a solution for diagnosis, and for planning and/or monitoring the course of an operation on the eye, with which an improved application is made possible by collecting additional information regarding the eye to be examined.

The proposed technical solution is not only provided for different ophthalmological apparatuses for diagnosis, and for planning and/or monitoring the course of operations on the eye, but also usable in other technical fields in which additional information of the object to be imaged is generated and evaluated accordingly as a result of using a digital camera suitable for recording HDR images.

It is particularly advantageous that the device may be designed as a retrofit unit for various ophthalmological apparatuses or else other apparatuses.

However, it is also possible to configure the device according to the invention as an independent one-function apparatus for retro-illumination analysis for the eye of the patient.

Various embodiments of systems, devices, and methods have been described herein. These embodiments are given only by way of example and are not intended to limit the scope of the claimed inventions. It should be appreciated. moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. & 112(f) are not to be invoked unless the specific terms “means for” or “step for” are recited in a claim.

1-22. (canceled)

23. A device that performs diagnosis, and planning, monitoring or both planning and monitoring of a course of an operation on an eye, the device comprising:
an optical system that fixates the eye with respect to an optical axis of the device;
the optical system that fixates the eye comprising a light source that projects a fixation mark into the eye, an image sensor that receives light reflected, diffracted or scattered by a cornea, a lens, and a retina of the eye:
a control unit configured to detect reflections, and light of the fixation mark diffracted and scattered by eye structures in images transmitted by the image sensor;
an high dynamic range (HDR) digital camera that records high dynamic range (HDR) images of the eye, wherein the digital camera is configured to record the HDR images of the eye predominantly only in a case of an activated fixation illumination, and
wherein the control unit is further configured to optimize and evaluate the HDR images recorded of the eye and to display the HDR images on a display unit.

24. The device as claimed in claim 23, wherein the control unit is configured to monitor alignment of the eye in relation to an optical axis of the device by detecting the fixation mark in images transmitted by the image sensor, and to display the alignment on a display.

25. The device as claimed in claim 23, wherein the HDR digital camera has an intensity depth or color depth selected from a group consisting of more than 8 bits, more than 10 to 14 bits and more than 16 bits.

26. The device as claimed in claim 23, wherein the HDR digital camera is a constituent part of a telecentric imaging system.

27. The device as claimed in claim 23, further comprising devices that suppress ambient light.

28. The device as claimed in claim 23, wherein the device is configured as a retrofit unit for ophthalmological apparatuses.

29. The device as claimed in claim 23, wherein the device is configured as an independent one-function apparatus for retro-illumination analysis of the eye of the patient.

30. A method of diagnosis, and of planning, monitoring or both planning and monitoring a course of an operation on an eye, the method comprising:
projecting a fixation mark into the eye with a light source;
imaging light of the fixation mark that is reflected, diffracted, or scattered by a comea, a lens, and a retina of the eye on an image sensor;
detecting, by application of a control unit, reflections and light of the fixation mark diffracted and scattered by eye structures in images transmitted by the image sensor to the control unit;
recording HDR images of the eye by an HDR digital camera predominantly only in a case of an activated fixation illumination and optimizing and evaluating the HDR images; and
displaying the HDR images on a display by operation of the control unit.

31. The method as claimed in claim 30, further comprising detecting the fixation mark in the images transmitted by the image sensor by application of the control unit, and thus monitoring alignment of the eye in relation to an optical axis of the device and displaying said alignment on a display.

32. The method as claimed in claim 30, further comprising recording HDR images of the eye by application of the digital camera with an intensity depth/color depth selected from a group consisting of more than 8 bits, 10 to 14 bits and 16 bits or more.

33. The method as claimed in claim 30, further comprising recording the HDR images of the eye telecentrically.

34. The method as claimed in claim 30, further comprising suppressing ambient light for a purpose of recording the eye images.

35. The method as claimed in claim 30, further comprising using known image processing and image optimization methods to optimize and evaluate the HDR images of the eye.

36. The method as claimed in claim 30, further comprising optimizing and evaluating the HDR images of the eye by:
a) removing image noise;
b) optimizing the intensity image representation to obtain a “moon-like” pupil image of the eye;
c) detecting a pupil edge and determining a diameter;
d) comparing the diameter with an expected value;
e) performing dilation should the diameter be too small and renewing performance of steps a) to d);
f) should the diameter at least correspond to the expected value, detecting image content in respect of abnormalities.

37. The method as claimed in claim 36, wherein detected abnormalities under step f) comprise shadow regions which indicate a cataract or posterior capsular opacification.

38. The method as claimed in claim 36, wherein detected abnormalities under f) comprise shadow regions which facilitate determination of a stage of cataract of the lens.

39. The method as claimed in claim 36, wherein detected abnormalities under f) represent a basis for further clinical examinations of the eye.

40. The method as claimed in claim 39, wherein detected abnormalities under f) represent a basis for an OCT-B-scan to be performed in accordance with the observed eye information, to determine the area/stage of the cataract and to obtain further clinically meaningful OCT depth information for the examined eye.

41. The method as claimed in claim 36, wherein detected abnormalities under f) also comprise the edge of an IOL, the markers thereof present or both, thereby facilitating deducing the position of the IOL the orientation of the IOL or both.

42. The method as claimed in claim 40, wherein detected abnormalities under f) comprise 3rd and 4th Purkinje reflexes, thereby facilitating determining tilt of an IOL, centration of the IOL or both in the eye.

43. The method as claimed in claim 36, wherein detected abnormalities under f) facilitate assessing size of a capsulorhexis quality of a capsulorhexis or both.

44. The method as claimed in claim 36, further comprising recording with the digital camera a series of HDR images of the eye, to facilitate monitoring a function of a tear film.
